(19) United States
(12) Patent Application Publication
(10) Pub. No.: US 2026/0325814 A1
(43) Pub. Date: Oct. 22, 2026

(54) CLEANSING AND CAPPING DEVICES FOR NEEDLEFREE VASCULAR ACCESS CONNECTORS

(71) Applicant: CleanSite Medical, Inc., Solana Bech, CA (US)

(72) Inventors: John GRANT, Solana Beach, CA (US); Daniel M. CHAMBERS, Solana Beach, CA (US); Richard A. PLUTH, San Diego, CA (US)

(73) Assignee: CleanSite Medical, Inc., Solana Beach, CA (US)

(21) Appl. No.: 19/704,276

(22) Filed: Jun. 10, 2026

Related U.S. Application Data
(63) Continuation-in-part of application No. 19/670,922, filed on May 7, 2026.
(63) Continuation-in-part of application No. 17/342,630, filed on Jun. 9, 2021.
(60) Provisional application No. 63/801,796, filed on May 7, 2025.
(60) Provisional application No. 63/821,429, filed on Jun. 10, 2025.

(51) Int. Cl. A61M 39/16; A61M 39/20

(52) U.S. Cl. CPC A61M 39/162; A61M 39/20; A61M 2209/084

(57) ABSTRACT

Capping and cleansing devices for capping and cleansing needlefree connectors, particularly luer access devices such as needlefree vascular access connectors, and methods for using said devices, are described. The devices of the invention each include interconnected first and second housings that a user can rotate independently, even after the device has been securely yet removably attached to, for example, the threaded valve region of a needlefree connector. Such rotation allows a compressible cleansing matrix secured in the device (preferably in a well in the second housing) to be used to actively cleanse potentially contaminated or soiled surface(s) of the valve region of a needlefree connector.

### RELATED APPLICATIONS

This application is a continuation-in-part of U.S. patent application Ser. No. 19/670,922, filed on 7 May 2026 (Docket No., CSM-0025-UT), which claims the benefit of and priority to U.S. provisional patent application Ser. No. 63/801,796 (attorney docket no. CSM-0025-PV2), filed 7 May 2025. This application also claims the benefit of and priority to commonly owned, U.S. provisional patent application No. 63/821,429, filed on 10 Jun. 2025 (Docket No. CSM-0022-PV13). This application is also a continuation-in-part of U.S. patent application Ser. No. 17/342,630, filed on 9 Jun. 2021 (Docket No. CSM-0022-CT), which is a continuation of U.S. Pat. No. 11,065,431, which issued from U.S. application Ser. No. 16/795,565, filed on 19 Feb. 2020 (Docket No. CSM-0022-UT), which claims the benefit of and priority to (now-expired) U.S. provisional patent application No. 62/807,239, filed on 19 Feb. 2019 (Docket No. CSM-0022-PV). and U.S. patent application Ser. No. 16/059,029, filed on 8 Aug. 2018 (Docket No. CSM-0021-UT) (to which U.S. Ser. No. 16/795,565 (Docket No. CSM-0022-UT) is a continuation-in-part), which claims the benefit of and priority to (now-expired) U.S. provisional patent application No. 62/542,770, filed on 8 Aug. 2017 (Docket No. CSM-0021-PV). All of the aforementioned priority applications are commonly owned, and each is hereby incorporated by reference, each in its entirety for any and all purposes.

### TECHNICAL FIELD OF THE INVENTION

This invention is directed to cleansing devices for cleansing and capping medical devices, particularly luer access devices such as needlefree, valved connectors (NFCs), and methods for making and using such articles.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

In the medical field, and in particular the area of infusion of fluids or aspiration of fluids to or from a patient, there remains a need to prevent the transmission of pathogens into or onto a patient from a potentially contaminated surface of a medical device such as a luer access device, for example, a needlefree, valved connector (NFC). Pathogens include microorganisms such as bacteria, fungi, and viruses, the transmission of which into a patient may result in an infection that could be life-threatening. Specific to healthcare settings, the term “nosocomial infection” describes those infections that originate from or occur in a hospital or hospital-like setting. In the U.S., nosocomial infections are estimated to occur in at least 5% of all acute care hospitalizations. The estimated incidence is more than two million cases per year, resulting in significant morbidity, mortality, and an expense. Indeed, nosocomial infections are estimated to more than double the mortality and morbidity risks of any admitted patient, and likely result in about 100,000 deaths a year in the United States alone. Common sites for the transmission of contaminating microorganisms into a patient's bloodstream are found on medical devices such as luer access devices, vials, needlefree (or needle free) valves, and the injection ports of vessels, tubing, and catheters. The incidence of such infections in patients is increasing, and infection control practitioners (ICPs) often cite improper cleansing of sites as a major source of such infections.

As described above, patient exposure to pathogens and infectious reagents (e.g., pathogenic bacteria, viruses, fungi, etc.) in medical settings (e.g., hospitals, out-patient surgery centers, home-care settings, etc.) is a matter of serious concern. One route of exposure to such reagents is the opening made in skin provided by the bore of needle, cannula, or other similar device used to provide access to a patient's vasculature. It is known that patients whose skin has been compromised in this way are at increased risk for developing serious blood stream infections. In the United States alone, approximately 300,000 blood stream infections per year result from the installation and use of peripheral intravenous catheters (PIVC), and more than 80,000 blood stream infections are associated with the use central venous catheters (CVC). All told, in the U.S. approximately 28,000 patients die annually from hospital-acquired infections that result from PIVC and CVC use, and many times that number are made seriously ill but survive. Costs associated with the care and treatment of patients that develop infections due to PIVC and CVC use is estimated to exceed $4 billion annually in the U.S. alone.

In hospital settings today, occupational health and safety regulations designed reduce the risk to health care workers from needle stick and similar injuries have resulted in the deployment of needlefree medical valves (also referred to herein as “needlefree connectors” or “NFCs”) whenever possible. Currently, more than 1 billion NFCs are used annually in hospitals throughout the U.S. Needlefree connectors are used primarily in conjunction with PIVC and CVC devices and associated IV administration and extension sets, which may contain from as few as one to as many as 3, 4, 5, or more NFCs. FIG. 2A includes an illustration of an example of a representative NFC in use today.

The widespread use of needlefree connectors in acute medicine has contributed to a marked increase in the incidence of hospital-acquired infections (HAIs), particularly blood stream infections (BSIs). To reduce the risk of infection from a needlefree connector contaminated with microorganisms, standard practice today requires that a nurse or other healthcare worker clean (or “scrub”) the surface of NFC by vigorously rubbing those of its exterior surfaces in the fluid path with a sterile alcohol swab or wipe immediately prior to making a fluid connection to the NFC, for example, by attaching a syringe to the NFC's threaded valve portion to deliver a medication via a PIVC already connected to the patient. Given the magnitude of the mortality and morbidity associated with HAIs, particularly with regard to central line-associated blood stream infections (CLABSIs), and the large number of blood stream infections that result from PIVC and CVC use (so-called “peripheral line-associated blood stream infections (PLABSIs) and central line-associated blood stream infections (CLABSIs), respectively), a long-recognized yet significant unmet need exists for articles or devices that can be used to reduce or eliminate the risk of initiating an HAI merely by accessing a patient's vasculature through a needlefree valve component of a PIVC or CVC inserted into a blood vessel of a patient.

Traditionally, and as noted above, cleaning, cleansing, or disinfecting a potentially contaminated NFC surface involved a protocol of alcohol swabbing prior to making the necessary connections to the site. Alcohol swabs are typically small pads of cotton gauze soaked in isopropyl alcohol (IPA), packed individually in foil packages to prevent evaporation of the IPA from the swab prior to use. Properly used, the package is opened at or near the site to be swabbed. With gloved hands, the swab is removed by a nurse or other healthcare provider and used to scrub the top and side surfaces of the valve portion of the NFC to be connected. After use, the swab and foil package are discarded and the cleansed valve portion of the NFC is allowed to dry, usually 20-30 seconds, immediately prior to making any connection. This “drying” period is important because, as the IPA dries, it breaks open the cellular walls of microorganisms, thereby killing them.

Unfortunately, because of increased duties and responsibilities, shrinking nursing staffs, and inadequate training, alcohol swabbing (or scrubbing) is often not performed or is poorly executed. A poorly swabbed site can carry microorganisms that, if allowed to enter a patient's body, can cause serious, and potentially life-threatening, infection. In addition, supervisory oversight is nearly impossible, because unless a supervisor actually observes swabbing as it is performed, the supervisor cannot know whether or not the scrubbing procedure was done properly or performed at all. Indeed, reported compliance with such “scrub the hub” protocols has been as low as 10%. Further, without at least a sufficient microscopic examination for microbial residue (e.g., biofilm), there may be no evidence of “scrubbing the hub” being performed.

Thus, a significant need still exists for devices and techniques cleanse sites on medical devices prior to their use with or connection to patients, and which eliminate technique-related and training issues and provide an unequivocal indicator that a site is clean prior to accessing a patient's vascular system.

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the singular forms “a”, “an”, and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The term “species”, when used in the context of describing a particular compound or molecule species, refers to a population of chemically indistinct molecules.

### SUMMARY OF THE INVENTION

The object of the invention is to address these long-standing but still unmet needs. This invention addresses these needs by providing patentable, single-use cleansing (disinfecting) and capping devices or articles that can be used to effectively and efficiently cleanse/disinfect and cap, and preferably sterilize, exposed surfaces of medical articles such as luer access devices, particularly needlefree connectors, particularly the accessible surface(s) of the threaded valve portions of needlefree connectors, particularly those surfaces (valve surfaces, threads, etc.) that may become contaminated with pathogens or other infectious reagents and which form part of the fluid communication pathway between an external fluid source (e.g., a medicine filled syringe with a male luer fitting, an IV bag, etc.) and a patient's blood stream. In the context of the invention, “cleanse” encompasses cleaning, disinfecting, sanitizing, and/or sterilizing, whereas “capping” refers to using a device, i.e., a “cap”, to cover a surface, or set of surfaces, of an NFC so as to limit or prevent exposure of such surface(s) to the environment (e.g., the air circulating in a hospital's intensive care unit, the microbiome resident on a patient's skin, clothing, bedding, unclean fingers, etc.) for a period longer than necessary to cleanse the desired surface(s) of a needlefree connector.

Thus, in one aspect, the invention provides capping and disinfecting devices for medical devices such as luer access devices, including needlefree, valved vascular access connectors (NFCs).

In general, such devices include at least two coupled housings, a first housing and a second housing, that can, when desired, be moved (e.g., rotated) independently of each other, and a compressible cleansing matrix preferably impregnated with a disinfectant, for example, a 70% IPA solution. The device also preferably includes an easily removable seal (lid) to maintain sterility and prevent loss of the disinfectant after the device is assembled until such time as it is used in the field, e.g., a hospital or other acute facility, a patient's home, a long-term care facility, etc.

In many preferred embodiments of this aspect, the first housing is an inner housing configured to allow the device to be screwed onto and unscrewed from the threaded valve portion of an NFC, and the second housing is an outer housing that retains the inner housing but which a user can, when desired, rotate independently of the inner housing to provide scrubbing or disinfecting action, and a compressible cleansing matrix preferably impregnated with a disinfectant, for example, a 70% IPA solution. The device also preferably includes an easily removable seal to maintain sterility and prevent loss of the disinfectant after the device is assembled until such time as it is used in the field to outer housing and cleanse an NFC.

As described above, many of the devices of the invention include a first, often an inner, housing. In some preferred embodiments, the first or inner housing is comprised of a sidewall that bounds a central, interior (preferably cylindrical) bore that spans between oppositely disposed first and second (or upper and lower, respectively) openings. In many of these embodiments, the first (upper) opening is sized to allow the compressible cleansing matrix resident at least in part in a matrix well or otherwise attached to a region of the inner surface of the second, often outer housing, to protrude into and through the opening into the first (e.g., inner) housing's central bore so that the compressible cleansing matrix can engage one or more exterior surfaces of a needlefree connector when the cleansing and capping device is secured to the connector. The second (lower) opening of the first (e.g., inner) housing is sized to allow the threaded valve portion of a needlefree connector to be cleansed and/or capped to be inserted into cleansing and capping device of the invention. The interior wall of the first (e.g., inner) housing's central bore includes one or more thread-engaging tabs or threads, preferably two (or more) oppositely disposed (or otherwise spaced) thread-engaging tabs, preferably near the lower opening. The thread-engaging tab(s) or threads is(are) configured to engage a complementary threaded region on the exterior surface of, for example, a needlefree connector such that the cleansing and capping device can, via association of the thread tabs or threads on the interior of the inner housing's central bore with complementary threads on the threaded portion of a needlefree connector, be securely threaded onto (or otherwise removably connected with) the targeted threaded portion of a needlefree connector for cleansing and/or capping or, if desired, both cleansing and capping. As will be appreciated, preferred thread designs, patterns, and the like conform to international medical device standards, for example, those that concern small-bore connectors used to combine different medical devices and accessories, for example, syringes, extension sets, IV sets, etc. and intravascular catheters such as single or multi-lumen central line catheters (including peripherally inserted central line catheters) and peripheral IV catheters.

In some preferred embodiments, the outer surface of the first (e.g., inner) housing includes a second (e.g., outer) housing-retaining region that includes one or more structures, for example, a circumferential flange (or spaced flange elements), that allow the first (e.g., inner) housing to be retained in the second (e.g., outer) housing via association with one or more complementary structures (e.g., a circumferential flange (or spaced flange elements) or other suitable engaging elements) on the inner surface of the sidewall of the second (e.g., outer) housing. Preferably, such configurations of complementary retaining elements also allow for smooth, low friction movement (i.e., rotation) of the first and second (e.g., inner and outer) housings in relation to each other during certain operations, for example, during a disinfection procedure for the threaded valve region a needlefree connector. In some of these embodiments, the retaining element(s) of the first (e.g., inner) housing can mechanically engage an adjacent region on the inner surface of the sidewall of the second (e.g., outer) housing, for example, when a user squeezes or otherwise applies sufficient force to the second (e.g., outer) housing to deform it so as to allow engaging regions on the inner surface of the second (e.g., outer) housing to engage corresponding engaging regions on the exterior surface of the first (e.g., inner) housing so as to allow the first and second (e.g., inner and outer) housings to rotate in unison (as would occur, for example, when a user attaches or removes a device according to the invention from an NFC).

In some of these embodiments, the retaining element(s) of the first (e.g., inner) housing can also serve as engaging elements with complementary regions, features, or structures on the inner surface of the sidewall of the second (e.g., outer) housing adjacent or otherwise in close proximity thereto. In other embodiments, the outer surface of the first (e.g., inner) housing further includes one or more second (e.g., outer) housing engaging elements or regions designed to associate with one or more first (e.g., inner) housing engaging elements or regions disposed on the interior or inner surface of the second (e.g., outer) housing. Examples of such elements include, for example, a circumferential band of spaced teeth or teeth-like elements protruding from the exterior surface of the first (e.g., inner) housing and positioned below the outer housing-retaining region (e.g., a circumferential flange), which teeth (or other suitable engaging structures) can be engaged by complementary structures arrayed on the interior surface of the second (outer) housing when the housings are assembled into a functional subassembly.

In other preferred embodiments, the upper exterior surface of the first (e.g., inner) housing includes a second (e.g., outer) housing-engaging region that includes one or more structures that allow the first (e.g., inner) housing to mechanically engage complementary structures (e.g., pawls or other suitable engaging elements) on the inner surface of the top of the second (e.g., outer) housing so that when the complementary engaging elements of the second and first (e.g., outer and inner) housings are brought into close proximity the engaging elements of the second (e.g., outer) housing and first (e.g., inner) housing engage, allowing the second and first (e.g., outer and inner) housings to rotate in unison. Certain preferred embodiments of second (e.g., outer) housing-engaging structures include spaced teeth (or other suitable engaging elements) arrayed on the top or upper surface of the first (e.g., inner) housing's preferably cylindrical sidewall. As will be appreciated, when such first and second (inner and outer) housing engaging elements are unmated or disengaged, a user can rotate or spin the second (e.g., outer) housing in relation to the first (e.g., inner) housing, as is, for example, done during a cleansing or disinfecting operation of a needlefree connector to which the device of the invention is attached. Thus, when the cleansing and capping device is secured to a needlefree connector, when such engaging elements are not functionally associated (or mated or otherwise engaged), a user can rotate the second (e.g., outer) housing (and compressible cleansing matrix) in relation to the first (e.g., inner) housing and connected needlefree connector. On the other hand, when the complementary elements on the inner surface of the top of the second (e.g., outer) housing and the upper surface of the top of the first (e.g., inner) housing are engaged (in whole or even partially), such as when a user applies downward pressure to the device to place it on or remove it from a needlefree connector, the first and second (e.g., inner and outer) housings rotate together, allowing, for example, the cleansing and capping device to be attached to or removed from the NFC.

In some preferred embodiments, the first (e.g., inner) housing and/or second (e.g., outer) housing also includes an NFC sealing member configured to provide a fluid tight seal between the cleansing and cleansing device of the invention and a needlefree connector connected thereto. In some embodiments the NFC sealing member is an O-ring, lip seal, or other comparable seal. In the case of, for example, an O-ring, it is preferably disposed in a channel formed in the inner surface of the wall of the first (inner) housing proximate to the second (lower) opening, typically below the thread-engaging tab(s)or threads of the device.

In embodiments wherein the first and second housings are configured as inner and outer housings, respectively, the devices of the invention also include an outer housing adapted or configured to retain the inner housing therein such that, when the device is attached to a needlefree connector, under certain conditions the outer housing can rotate (preferably about its central axis) in relation to the inner housing. Any suitable configuration of complementary mechanical or structural features or elements on facing or opposing surfaces can be used to provide retention of the inner housing inside the outer housing's main cavity and to allow for engagement and disengagement of the outer housing from the inner housing in order to allow the outer housing to be rotated in relation to the inner housing when the device is attached to a needlefree connector and a user desires to cleanse the corresponding surface(s) of the NFC using the cleansing and capping device of the invention.

In some of these embodiments, when the device of the invention is attached to a needlefree connector, the first and second (inner and outer) housings adopt a disengaged, neutral, or rotating configuration relative to each other such that a user can rotate the second (e.g., outer) housing in relation to the first (e.g., inner) housing to perform a cleansing operation on the valve portion of the NFC to which the device is attached. Such a disengaged, neutral, or rotating configuration can be achieved by any suitable approach, including by providing complementary engaging elements or structures on adjacent surfaces of the inner and outer (first and second) housings that under certain conditions, for example, when the outer (second) housing is pulled up, pushed down, or squeezed by a user in relation to the inner (first) housing, engage each other; otherwise the engaging elements remain disengaged, which allows rotation of the outer housing in relation to the inner housing when the device is secured to an NFC. Features that allow transitioning between engaged and disengaged positions include springs or biasing or resilient elements or materials. In other embodiments, when the device is attached to a needlefree connector, the inner and outer (first and second) housings adopt an engaged configuration relative to each other such that they rotate in unison unless a user applies sufficient force to the outer housing to cause the engaging elements to disengage and thus allow the outer housing to be rotated independently of the inner housing.

The outer (second) housing includes a cylindrical cavity designed to receive and retain the inner (first) housing using one or more features or elements that allow the outer (second) housing to be rotated in relation to the inner (first) housing if and when desired. The cavity is formed by a curved outer sidewall that in some embodiments is joined to a top portion of the outer (second) housing about its periphery and which also preferably has a concentric central matrix well or matrix attachment region to or with which the compressible cleansing matrix is attached or otherwise associated, although in some embodiments some degree of eccentricity between the matrix well and central rotational axis of the outer housing may be desired. In some embodiments, the outer (second) housing is formed by a sidewall that is tapered and/or has one or more steps.

In some preferred embodiments, the inner surface of the top of the outer (second) housing includes one or more inner (first) housing engaging elements or structures (e.g., teeth) designed to releasably engage complementary structures in the outer (second) housing-engaging region on the top of the inner (first) housing. Engagement of the outer (second) housing's inner (first) housing engaging structure(s) with those in the outer (second) housing-engaging region of the inner (first) housing allow a user to rotate the outer and inner (second and first) housings in unison, for example, as a cleansing and capping device's inner (first) housing is screwed onto the threaded portion of a needlefree connector to be cleansed and/or capped. Once the device is releasably secured to a needlefree connector via the inner (first) housing, the outer (second) housing's inner (first) housing engaging elements or structure(s) can be (or are) disengaged from the outer (second) housing-engaging elements of the inner (first) housing, for example, by the biasing action or resilience of a the compressible cleansing matrix, thereby allowing a user to rotate the outer (second) housing about its central axis in relation to the inner (first) housing. A representative example of such engaging structures is shown in published US patent application publication no. 2018/0304067, although features such as an inner housing having an opening in its top to allow a compressible cleansing matrix attached to the inner surface of the top of the outer (second) housing to extend into the bore of the inner (first) housing so that it can contact surfaces of a needlefree connector upon connection of the former to the latter are also envisioned.

In some of these embodiments, the outer (second) housing may include one or more vents to allow fluid and/or air from inside the device to escape as the cleansing and capping device is secured to a needlefree connector, while in other embodiments, no vent(s) is(are) provided. In embodiments with one or more vents, a membrane, filter, or other permeable or semi-permeable barrier may be employed to allow a unidirectional or bidirectional flow of air, gas, or vapor through the vent(s) but prevent the movement of microorganisms (e.g., bacteria, fungi, viruses, etc.) into the cleansing and capping device of the invention.

In certain preferred embodiments, the outer surface of at least the outer (second) housing of a cleansing and capping device according to the invention, and in those embodiments where both the first and second housings have outer surfaces at least a portion of which are accessible to and are intended for user interaction (e.g., grasping or gripping to rotate the device and/or housing(s)), one, or advantageously both, of the first and second housings include one or more grip-enhancing coatings, structures (e.g., a plurality of vertical ridges), surfaces (e.g., a textured surface), or other features. Such grip-enhancing features facilitate a user's grasp of the housing of a cleansing and capping between her/his fingers, which can be helpful not only during insertion and removal of a needlefree connector into or from the cleansing and capping device, but also during the cleansing process, where the user rotates the second (e.g., outer) housing in relation to the first (e.g., inner) housing in order to scrub and thereby clean or cleanse the surface(s) of the inserted needlefree connector with the compressible cleansing matrix of the device.

In some preferred embodiments, the devices of the invention include one or more elements or features arrayed on facing surfaces of the inner and outer (first and second) housings that allow a user to sense that the outer (second) housing is rotating in relation to the inner (first) housing in order to provide cleansing action on the valve surface of the NFC to which the device is attached. Such sensory feedback (or haptics) can include one or more of auditory, tactile, and/or visual stimuli generated from the device by rotation of the outer (second) housing in relation to the inner (first) housing.

In the devices of the invention, the first and second (e.g., inner and outer) housings are advantageously manufactured separately by any suitable process, for example, 3D-printing, injection molding, etc., and then assembled into a multi-part (e.g., two-part) subassembly in which the first (e.g., inner) housing is retained within the main cavity of the second (e.g., outer) housing by one or more complementary retaining elements, features, or structures on each housing.

In many embodiments, the first and second (e.g., inner and outer) housings also include complementary mechanical or structural engaging elements, features, or structures on one or more interfacing surfaces that can be engaged and disengaged so as to allow the first and second (e.g., inner and outer) housings to rotate together or to allow the second (e.g., outer) housing to rotate independently of the first (e.g., inner) housing. In this way, the first and second (e.g., inner and outer) housings can be associated such that they can rotate in unison, allowing a user to thread (or screw) the device onto or remove (unscrew) it from the threaded valve portion of an NFC if and when desired, while also making it possible for a user to rotate the second (e.g., outer) housing in relation to the first (e.g., inner) housing, thereby allowing the compressible cleansing matrix to effectively scrub or cleanse the region(s) of a threaded valve portion of an NFC to which it is attached. In certain preferred embodiments, the first and second (e.g., inner and outer, respectively) housings further include complementary mechanical or structural housing sealing elements, features, or structures on one or more interfacing surfaces that allow formation of seal between adjacent surfaces of the first and second (e.g., inner and outer) housings, which seal is preferably substantially fluid tight but does not substantially hinder or inhibit rotation of the second (e.g., outer) housing in relation to the first (e.g., inner) housing during performance of a cleansing procedure or process by a user. In some embodiments, the first (e.g., inner) housing may also include a seal that interacts with the needlefree connector to form an additional or alternate seal.

In other embodiments, the first and second housings also include complementary mechanical or structural engaging elements, features, or structures on one or more interfacing surfaces that allow them to be associated during assembly such that, after assembly, the first and second housings can be moved independently of each other (e.g., rotated) but without the need for them to be moved in unison (e.g., be engaged) in order to attach or detach (e.g., thread or unthread) the device from an NFC. For example, in some embodiments the first and second housing at all times retain the ability for a user to move (e.g., rotate) one or both of the first and/or second housings in relation to each other, such as may occur in certain embodiments where the first housing is configured for NFC attachment while the second housing, while operably associated with the first housing, is also configured to retain the compressible cleansing matrix such that when the second housing is rotated by a user after the device is connected (via the first housing) to an NFC, the surface(s) of the NFC in contact with the compressible cleansing matrix can be cleansed (e.g., disinfected) by rotating the second housing, which rotation moves the second housing and compressible cleansing matrix in relation to the first housing and NFC.

As described, a cleansing and capping device of the invention also includes a compressible cleansing matrix disposed therein. In most embodiments, the compressible cleansing matrix is disposed in a matrix well or the like in the interior of the second (e.g., outer) housing, although any suitable retaining configuration can be employed that allows the compressible cleansing matrix to rotate in conjunction with rotation of the second (e.g., outer) housing so as to provide the capability of using the compressible cleansing matrix to scrub or otherwise clean, cleanse, or disinfect the surface(s) of the valve region of a needlefree connector. As will be appreciated, the compressible cleansing matrix is positioned to contact one or more exterior surface(s) of an NFC connected to a cleansing and capping device of the invention. The compressible cleansing matrix, for example, an open-cell or felted foam, can advantageously be retained in the matrix well by one or more matrix retaining elements, which element(s) assist in retention of the compressible cleansing matrix in the matrix well in addition to transmission of rotational forces from the second (e.g., outer) housing to the compressible cleansing matrix as occurs during a procedure to disinfect or cleanse a needlefree connector. As will be appreciated, during such rotation (of the second (e.g., outer) housing and compressible cleansing matrix), the compressible cleansing matrix also rotates in relation to the first (e.g., inner) housing when the outer housing is rotated during a procedure to disinfect or cleanse a needlefree connector. The compressible cleansing matrix attached to or otherwise associated with the second (e.g., outer) housing can be axially compressed, i.e., compressed along the central axis of the second (e.g., outer) housing's matrix well, upon insertion of a needlefree connector, advantageously, its threaded valve region, into such a cleansing and capping device.

Because the needlefree connector surface(s) to be cleansed may be contaminated with microorganisms that form a biofilm (i.e., a matrix of microorganisms and extracellular material attached to a surface, which enables the microorganisms, typically bacteria and/or fungi, to adhere to a surface and carry out certain biochemical processes), the compressible cleansing matrix also preferably has sufficient mechanical integrity when compressed and rotated to allow it to disrupt any biofilm that may be present on the surface of the needlefree connector, as can occur by rotating, twisting, or otherwise moving the then-compressed cleansing matrix in relation to the needlefree connector, for example, by rotating the outer housing (to which the compressible cleansing matrix is attached) in relation to the inner housing of the cleansing and capping device and the needlefree connector to which inner housing is releasably attached. The resulting friction between the compressed cleansing matrix and surface of the needlefree connector disrupts the biofilm, thereby cleansing, and preferably sterilizing, the needlefree connector. Leaving the cleansing and capping device secured to (i.e., capping) the needlefree connector after such a cleansing operation will limit, and preferably preclude, biofilm regrowth and/or the microbial recolonization of cleansed surfaces (which remain in contact with the compressible cleansing matrix).

In preferred embodiments, the compressible cleansing matrix includes one or more cleansing reagent species dispersed therein, preferably at the time the device is manufactured, although in some embodiments, the cleansing reagent may be dispersed into the matrix just prior to the matrix coming into contact with a needlefree connector. In embodiments of the latter sort, the cleansing reagent is preferably housed in the housing of the cleansing and capping device in a reservoir configured to be ruptured just prior to performance of a cleansing operation. In some embodiments, the cleansing and capping device of the invention will include a valve, vent, or other opening to allow liquid in the cleansing reagent to evaporate.

In some preferred embodiments, the compressible cleansing matrix includes two or more components. In some of such embodiments, one component of the matrix is attached to the inner surface of the second (e.g., outer) housing and another component is secured to the inner surface of the wall forming the first (e.g., inner) housing, preferably between protruding threaded regions adapted to engage complementary threads on a needlefree connector. If present, the component(s) of the compressible cleansing matrix secured to the inner surface of the first (e.g., inner) housing wall is(are) preferably configured to radially compress upon association with a needlefree connector to be capped and cleansed.

In preferred embodiments, the cleansing and capping devices of the invention include a removable lid or seal attached to the second (e.g., outer) housing to seal the device, thus separating the interior spaces and structures of the first and second (e.g., inner and outer) housings from the external environment. Such a lid or seal prevents exposure of the device's interior, including the first (e.g., inner) housing and compressible cleansing matrix, to the environment until the removable (preferably, peelable) lid or seal is removed, typically by a healthcare worker just prior to her/his use of the cleansing and capping device to clean, cleanse, or disinfect a needlefree connector to which a fluid connection is to be made. In preferred embodiments, such cleansing substantially disrupts any microbial contamination, for example, microbial biofilm or other microbial contamination that may exist on surfaces contacted by the compressible cleansing matrix. If desired, the cleansing and capping device can be left in place (typically after cleansing the needlefree connector attached thereto) in order to cap the needlefree connector until it is further accessed, thereby minimizing exposure of capped exterior surfaces of the NFC to potential pathogen contamination (and biofilm formation) from the surrounding environment. Lids or seals are typically installed during manufacture of a cleansing and capping device of the invention. In those embodiments where the cleansing and capping devices are sterilized during manufacture (e.g., by irradiation, exposure to ethylene oxide, etc.), lids or seals are preferably applied prior to packaging and sterilization.

In some preferred embodiments, the devices of the invention are sealed individually, while in other embodiments, 2-20 or more devices are sealed onto a single piece of lidding or sealing stock, after which they may be separated into individual sealed products or maintained in strip form, as a strip format having multiple devices all sealed to a single strip is a convenient format for use in healthcare environments, where such strips can be hung, for example, from an IV pole at a patient's bedside. As will be appreciated, two or more devices of the invention that are individually sealed can also be provided in a strip format, in a bag or box, in any other suitable format convenient for users at a patient's bedside. After sealing and packaging, the devices of the invention are sterilized using any suitable sterilization method (e.g., gamma or e-beam irradiation, treatment with ethylene oxide, etc.) compatible with the materials used to manufacture the particular device(s) of the invention.

Other aspects of the invention concern methods of cleansing and/or capping needlefree connectors using a cleansing and capping device according to the invention. Such methods typically involve disengaging the engaging elements of the second (e.g., outer) housing and first (e.g., inner) housing after it has been connected to a needlefree connector, thus allowing a user to rotate or spin the second housing in relation to the first housing and needlefree connector to which the device of the invention is secured. Such disengagement does not impair contact between the device's compressible cleansing matrix and the associated surface(s) of the needlefree connector intended to be cleansed. Spinning or rotation of the second (e.g., outer) housing in relation to the first (e.g., inner) housing, and the associated surface(s) of the needlefree connector, allow those surfaces to be scrubbed, thereby cleansing them. Preferably, such cleansing methods provide for the disruption of any biofilm present on the surface(s) of the needlefree connector associated the cleansing and capping device. And in those embodiments where the compressible cleansing matrix contains one or more antimicrobial reagents, microbes and pathogens present in such biofilm and/or on such surface(s) are preferably destroyed or rendered nonviable. In other words, the devices of the invention can be effectively used to remove contamination and various contaminants (including microbial biofilms and the microorganisms that form such biofilms), and the disinfectant in the compressible cleansing matrix can kill or render inert some or all of the microorganisms exposed to the disinfectant.

Features and advantages of the invention will be apparent from the following detailed description, and appended claims.

### BRIEF DESCRIPTION OF THE DRAWINGS

These and other aspects will now be described in detail with reference to the following drawings. Unless otherwise indicated, it is understood that the drawings are not to scale, as they are intended merely to facilitate understanding of the invention as opposed to specific dimensions, etc. In the drawings, like numbers in two or more drawings represent like elements.

FIG. 1 shows three drawings, A, B, and C, of a representative cleansing and capping device of the invention, its constituent parts (A), and two views of the device associated with a needlefree connector (B and C).

FIG. 2 contains five drawings, A-E. FIGS. 2A-C show different views of an assembled, sealed representative cleansing and capping device of the invention, and FIGS. 2D and 2E show that device operably connected to a needlefree connector.

FIG. 3 contains 10 drawings, A-J, that show four views of the second (upper) housing (A-D) and six views (E-J) of the lower housing of a representative cleansing and capping device of the invention.

FIG. 4 contains 7 drawings, A-G, that show seven cross-section views looking up from below at the inner and outer (second and first, respectively) housings of a representative cleansing and capping device of the invention threaded onto a needlefree connector.

The details of one or more embodiments are set forth in the accompanying drawings and the description below. Other features and advantages will be apparent from the description and drawings, and from the claims.

### DETAILED DESCRIPTION

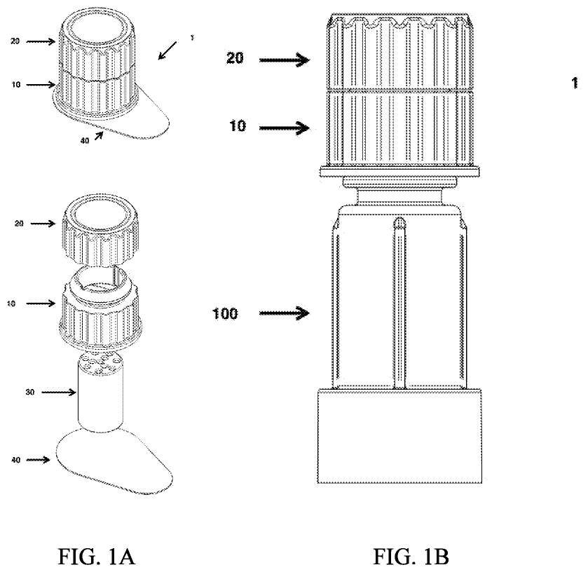

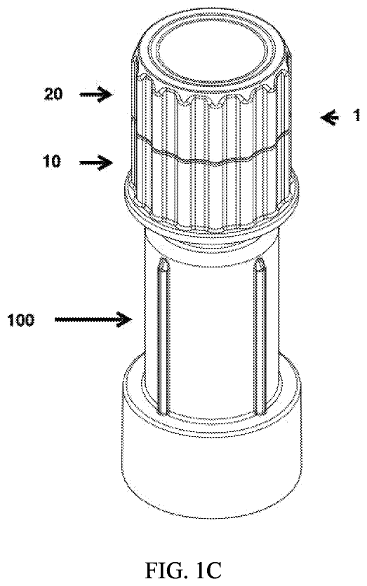

In the following detailed description, reference is made to the accompanying figures (FIGS. 1-4), which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here.

This invention concerns patentable single-use cleansing and capping devices that can be used to effectively and efficiently clean, disinfect, and preferably sterilize, exposed surfaces of needlefree connectors, particularly those of luer access devices such as needlefree medical valves that at times become part of a fluid communication pathway for introduction of fluids (e.g., IV fluids, blood, plasma, medicines, etc.) into a patient, as these surfaces are at risk for contamination with pathogens and infectious reagents such as bacteria, fungi, and viruses. “Single-use” (or “single purpose”) refers to an article or device suitable for one use or purpose only, as distinguished from “dual” or “multiple” use or purpose devices. Thus, in the context of the invention, a “single-use” cleansing and capping device is one that is useful for cleansing, for example, a needlefree medical valve. After the cleansing operation, the device may, if desired, be left in place on the needlefree connector until a subsequent fluid connection is made in order to prevent recontamination of the connector's cleansed surface(s), as would occur if the cleansing and capping device of the invention was removed immediately following “scrubbing” of the connector without a fluid connection then being made. After removal, the device of the invention is preferably discarded. Prior to removal, however, the cleansing and capping device can again be used to cleanse the capped surface(s) of the needlefree connector.

In general, a cleansing and capping devices of the invention include a first, often an inner, housing configured to allow the device to be screwed onto and unscrewed from the threaded valve portion of an NFC, a second, often an outer, housing that retains the first (e.g., inner) housing but which a user can, when desired, rotate independently of the first (e.g., inner) housing to provide scrubbing or disinfecting action, and a compressible cleansing matrix preferably impregnated with a disinfectant, for example, a 70% IPA solution. The device also preferably includes an easily removable seal to maintain sterility and prevent loss of the disinfectant after the device is assembled until such time as it is used in the field to cleanse and/or cap an NFC.

As described, the devices of the invention include a first, often an inner, housing. In some preferred embodiments, the first housing is comprised of a sidewall that bounds a central, interior (preferably cylindrical) bore that spans between oppositely disposed first and second (or upper and lower, respectively) openings. In many of these embodiments, the first (upper) opening is sized to allow the compressible cleansing matrix resident at least in part in a matrix well or otherwise attached to the inner surface of the second (e.g., outer) housing to protrude into and through the opening into the first housing's central bore so that the compressible cleansing matrix can engage one or more exterior surfaces of a needlefree connector when the capping and cleaning device is secured to the connector. The second (lower) opening of the first (often inner) housing is sized to allow the threaded valve portion of a needless connector to be capped and/or cleansed to be inserted into cleansing and capping device of the invention. The interior wall of the first housing's central bore includes one or more thread-engaging tabs or threads (often helical), preferably two (or more) oppositely disposed (or otherwise spaced) thread-engaging tabs or threads, preferably near the lower opening. The thread-engaging tab(s) or thread(s) is(are) configured to engage a complementary threaded region on the exterior surface of, for example, a needlefree connector such that the cleansing and capping device can, via association of the thread tabs or threads on the interior of the first housing's central bore with complementary threads on the threaded portion of a needlefree connector, be securely threaded onto (or otherwise removably connected with) the targeted threaded portion of a needlefree connector for capping and, if desired, cleansing.

In some preferred embodiments, the outer surface of the first (e.g., inner) housing includes a second (e.g., outer) housing-retaining region that includes one or more structures, for example, a circumferential flange (or spaced flange elements), that allow the inner housing to be retained in the outer housing via association with one or more complementary structures (e.g., a circumferential flange (or spaced flange elements) or other suitable engaging elements) on the inner surface of the sidewall of the second (e.g., outer) housing. Preferably, such configurations of complementary retaining elements also allow for smooth, low friction movement (for example, rotation) of the second and first (e.g., outer and inner) housings in relation to each other during certain operations, for example, during a disinfection procedure of a needlefree connector, when the device is being threaded onto the threaded valve portion of a needlefree connector, etc. In some of these embodiments, the retaining element(s) of the first (e.g., inner) housing can mechanically engage an adjacent region on the inner surface of the sidewall of the second (e.g., outer) housing, for example, when, in some embodiments, a user squeezes or otherwise applies sufficient force to the outer housing to deform it so as to allow engaging regions on the inner surface of the outer housing to engage corresponding engaging regions on the exterior surface of the first (here, inner) housing so as to allow the inner and outer housings to rotate in unison as would occur, for example, when a user attaches or removes a device from an NFC. In some of these embodiments, the retaining element(s) of the inner housing can also serve as engaging elements with complementary regions, features, or structures on the inner surface of the sidewall of the outer housing adjacent or otherwise in close proximity thereto.

In many embodiments, the outer surface of the first (e.g., inner) housing further includes one or more second (e.g., outer) housing engaging elements or regions designed to associate with one or more a first (e.g., inner) housing engaging elements or regions disposed on the interior or inner surface of the second (e.g., outer) housing. Examples of such elements include, for example, a circumferential band of spaced teeth or teeth-like elements protruding from the exterior surface of the first (e.g., inner) housing and positioned below the second (e.g., outer) housing-retaining region (e.g., a circumferential flange), which teeth (or other suitable engaging structures) can be engaged by complementary structures arrayed on the interior surface of the outer housing when the housings are assembled into a functional subassembly.

In other preferred embodiments, the upper exterior surface of the first (e.g., inner) housing includes an outer housing-engaging region that includes one or more structures that allow the inner housing to mechanically engage complementary structures (e.g., pawls or other suitable engaging elements) on the inner surface of the top of the second (e.g., outer) housing so that when the complementary engaging elements of the first and second (e.g., inner and outer) housings are brought into close proximity, the engaging elements of the second and first housings engage, allowing the housings to rotate in unison. Certain preferred embodiments of second (e.g., outer) housing-engaging structures include spaced teeth (or other suitable engaging elements) arrayed on the top or upper surface of the first (e.g., inner) housing's preferably cylindrical sidewall. As will be appreciated, when such first and second (e.g., inner and outer) housing engaging elements are unmated or disengaged, a user can rotate or spin the second (outer) housing in relation to the first (inner) housing, as is, for example, done during a cleansing or disinfecting operation of the needlefree connector to which the device of the invention is attached. Thus, when the cleansing and capping device is secured to a needlefree connector, when such engaging elements are not functionally associated (or mated or otherwise engaged), a user can rotate the second (e.g., outer) housing (and compressible cleansing matrix operably connected thereto) in relation to the first (e.g., inner) housing and connected needlefree connector. On the other hand, in such embodiments, when the complementary elements on the inner surface of the top of the second (outer) housing and the upper surface of the top of the first (inner) housing are engaged (in whole or even partially), such as when a user applies downward pressure to the device to place it on or remove it from a needlefree connector, the inner and outer housings rotate together, allowing, for example, the cleansing and capping device to be attached to or removed from the NFC.

In still other embodiments, the cleansing and capping devices of the invention include upper (second) and lower (first) housings include one or more structures (e.g., a channel or other suitable retaining element) that allow the lower housing to mechanically retained by complementary structures (e.g., a housing connecting flange or other suitable engaging element) on the inner surface at or near the bottom of the main cavity of the second (e.g., outer) housing so that when the complementary retaining elements of the upper and lower (e.g., second and first, respectively) housings are operably associated, the retaining elements of the housings engage, preferably allowing the housings to rotate independently of one another about an axis defined by their central bores when attached to other.

Advantageously, one or both of the first (e.g., inner or lower) and/or second (e.g., outer or lower) housing also includes an NFC sealing member configured to provide a fluid tight seal between the cleansing and capping device of the invention and a needlefree connector connected thereto. In some embodiments the NFC sealing member is an O-ring (or comparable seal) preferably disposed in a channel formed in the inner surface of the wall of the inner housing proximate to the second (lower) opening, typically below the thread-engaging tab(s)(or threads).

In some embodiments, the devices of the invention also include an outer housing adapted or configured to retain the inner housing therein such that, when the device is attached to a needlefree connector, under certain conditions the outer housing can rotate (preferably about its central axis) in relation to the inner housing. Any suitable configuration of complementary mechanical or structural features or elements on facing or opposing surfaces can be used to provide retention of the inner housing inside the outer housing's main cavity and to allow for engagement and disengagement of the outer housing from the inner housing in order to allow the outer housing to be rotated in relation to the inner housing when the device is attached to a needlefree connector and a user desires to cleanse the corresponding surface(s) of the NFC using the cleansing and capping device of the invention.

In other embodiments, the devices of the invention include a first (e.g., lower) housing adapted or configured to retain the second (e.g., upper) housing such that, when the device is attached to a needlefree connector via the one or more threads (or one or more thread tabs) in the central bore of the lower housing, advantageously the second (upper) housing can rotate (preferably about its central axis) in relation to the first (lower) housing. Any suitable configuration of complementary mechanical or structural features or elements on facing or opposing surfaces can be used to provide retention of the second (upper) housing with the first (lower) housing and to allow for the second (upper) housing to be rotated in relation to the first (lower) housing when the device is attached to a needlefree connector and a user desires to cleanse the corresponding surface(s) of the NFC using the cleansing and capping device of the invention.

In some embodiments, when a device of the invention is attached to a needlefree connector, the first and second (e.g., inner and outer) housings adopt a disengaged, neutral, or rotating configuration relative to each other such that a user can rotate the outer housing in relation to the first (e.g., inner) housing to perform a cleansing operation on the valve portion of the NFC to which the device is attached. Such a disengaged, neutral, or rotating configuration can be achieved by any suitable approach, including by providing complementary engaging elements or structures on adjacent surfaces of the first and second (e.g., inner and outer) housings that under certain conditions, for example, when the second (e.g., outer) housing is pulled up, pushed down, or squeezed by a user in relation to the first (e.g., inner) housing, engage each other; otherwise the engaging elements remain disengaged, which allows rotation of the second (outer) housing in relation to the first (inner) housing when the device is secured to an NFC. Features that allow transitioning between engaged and disengaged positions include springs or biasing or resilient elements or materials. In other embodiments, when the device is attached to a needlefree connector, the first and second (e.g., inner and outer) housings adopt an engaged configuration relative to each other such that they rotate in unison unless a user applies sufficient force to the second (e.g., outer) housing to cause the engaging elements to disengage and thus allow it to be rotated independently of the first (e.g., inner) housing.

The second (e.g., outer or upper) housing advantageously includes a preferably cylindrical cavity designed to receive and retain the first (e.g., inner or lower) housing using one or more features or elements that allow the second housing to be rotated in relation to the first housing if and when desired. The cavity is formed by a curved outer sidewall that in some embodiments is joined to a top portion of the housing about its periphery and which also preferably has a concentric central matrix well or matrix attachment region to or with which the compressible cleansing matrix is attached or otherwise associated, although in some embodiments some degree of eccentricity between the matrix well and central rotational axis of the second housing may be desired. In some embodiments, the second housing is formed by a sidewall that is tapered and/or has one or more steps. The outer diameter of the second housing can be substantially the same as that of the lower housing, although in some embodiments the outer diameter of the second (e.g., upper) housing can be smaller than the outer diameter of the first (e.g., lower) housing. In embodiments that do not require that the first and second housings be engaged so as to rotate in unison to achieve attachment to or removal from a needlefree connector, a device having a first housing with a larger outer diameter (or circumference) than that of the second (upper) housing can, for example, make the device easier for a user to hold and attach to a needlefree connector.

In some preferred embodiments, the inner surface of the top of the second (outer) housing includes one or more inner housing engaging elements or structures (e.g., teeth) designed to releasably engage complementary structures in the second housing-engaging region on the top of the first (inner) housing. Engagement of the outer housing's inner housing engaging structure(s) with those in the outer housing-engaging region of the inner housing allow a user to rotate the outer housing and inner housing in unison, for example, as a cleansing and capping 's inner housing is screwed onto the threaded portion of a needlefree connector to be cleansed and/or capped. Once the device is releasably secured to a needlefree connector via the inner housing, the outer housing's inner housing engaging elements or structure(s) can be (or are) disengaged from the outer housing-engaging elements of the inner housing, for example, by the biasing action or resilience of a the compressible cleansing matrix, thereby allowing a user to rotate the outer housing about its central axis in relation to the inner housing. Features such as an inner housing having an opening in its top to allow a compressible cleansing matrix attached to the inner surface of the top of the outer housing to extend into the bore of the inner housing so that it can contact surfaces of a needlefree connector upon connection of the former to the latter are also envisioned.

In some of embodiments, the second (e.g., outer) housing may include one or more vents to allow fluid and/or air from inside the device to escape as the cleansing and capping device is secured to a needlefree connector, while in other embodiments, no vent(s) is(are) provided. In embodiments with one or more vents, a membrane, filter, or other permeable or semi-permeable barrier may be employed to allow a unidirectional or bidirectional flow of air, gas, or vapor through the vent(s) but prevent the movement of microorganisms (e.g., bacteria, fungi, viruses, etc.) into the cleansing and capping device of the invention.

In certain preferred embodiments, the outer surface of the first and/or second housings of a cleansing and capping device according to the invention includes one or more grip-enhancing structures (e.g., a plurality of vertical ridges, ribs, bumps, and/or the like) or coatings. Such grip-enhancing structures or coatings facilitate a user's grasp of a particular housing of a cleansing and capping device between her/his fingers, which can be helpful not only during attachment and removal of a needlefree connector from the capping cleansing device, but also during the cleansing process, where, for example, in many embodiments the user rotates the second (e.g., outer) housing in relation to the first (e.g., inner) housing in order to scrub and thereby clean or cleanse the surface(s) of the attached needlefree connector with the compressible cleansing matrix of the device. In embodiments where a user can grasp both the first and second housings, as is the case, for example, in many embodiments that include upper and lower housings, part or all of the exterior surface of the housings accessible to a user can include such grip-enhancing structures or coatings.

In some preferred embodiments, the devices of the invention include one or more elements or features arrayed on facing surfaces of the first and second housings that allow a user to sense that the second (e.g., outer or upper) housing is rotating in relation to the first (e.g., inner or lower) housing in order to provide cleansing action on the valve surface of the NFC to which the device is attached. Such sensory feedback can include one or more of auditory, tactile, and/or visual stimuli generated from the device by rotation of one housing in relation to another of the device's housings.

In some of these embodiments, the devices of the invention comprise a resilient inner body disposed within a cap that can be rotated or turned in relation thereto when the cleansing and capping device is secured to a needlefree connector, and a compressible matrix element containing one or more antimicrobial reagents and having a structure to allow capped surfaces of the needlefree connector to be cleansed. More specifically, the resilient inner body has a wall that forms a central, interior (preferably cylindrical) bore that extends between oppositely disposed first and second (or upper and lower, respectively) openings. The interior wall of the central bore nearer the second (lower) opening includes one or more thread-engaging tabs (or threads), preferably two thread-engaging tabs disposed opposite to each other. The thread-engaging tab(s) (or threads) is(are) are configured to engage a complementary threaded region on the exterior surface of, for example, a needlefree medical valve. This allows the cleansing and capping device to be securely threaded onto the targeted threaded portion of a needlefree connector for cleansing and, if desired, capping that portion of the needlefree connector. The exterior surface of the resilient inner body includes one or more structures that allow it to mechanically engage and disengage complementary structures disposed on an inner surface of the cap.

In many embodiments, the resilient inner body's exterior surface includes a cap-engaging region that includes one or more structures such as spaced protrusions (e.g., teeth) that allow the resilient inner body to mechanically engage and disengage complementary engaging structures on an interior surface of the cap. Preferred embodiments of cap-engaging structures include alternating teeth and grooves (or channels) arrayed about the outer circumference of the resilient inner body, which teeth and grooves are complementary to one or more spaced engaging structures (e.g., teeth) disposed on the inner surface of the cap's outer wall. In other embodiments, the cap-engaging region of the resilient inner body is disposed on its inner surface for engagement with one or more complementary engaging structures (e.g., teeth) arrayed on the exterior or outer surface of matrix well wall. As will be appreciated, in configurations that include teeth and channels, the “teeth” can be raised protrusions and the “channels” can be the spaces or gaps between the raised protrusions.

The resilient inner body also includes a compressible region. In many preferred embodiments, it is located above the thread-engaging tab(s)(or threads) and cap-engaging region. The compressible region can be any structure that allows the resilient inner body to be compressed so as to bring the first and second openings closer together to allow disengagement of the engaging structures of the cap and resilient inner body. In preferred embodiments, the compressible region is a torsion spring, which in certain particularly preferred embodiments is a molded torsion spring formed from a plastic or other sufficiently flexible or resilient material, preferably during injection molding, as part of the resilient inner body.

In some embodiments, the resilient inner body is made from two or parts that are then assembled to form the complete inner body. For example, a resilient inner body can be formed as two or more separate parts that are assembled, one on top of the other, during manufacture of the cleansing and capping device of the invention. For instance, in embodiments where the resilient inner body is made of two parts, the upper part preferably comprises the compressible region (e.g., a torsion spring formed during injection molding of the upper part), while the lower part comprises the cap-engaging region and the thread-engaging tab(s)(or threads). In contrast, a representative three-part embodiment of a resilient inner body includes an upper section that comprises the first (upper) opening and the compressible region, a midsection that comprises the cap-engaging region, and a lower section that includes the thread-engaging tab(s)(or threads) and second (lower) opening. In an alternative three-part embodiment, the upper section comprises the first (upper) opening and cap-engaging region, the midsection comprises the compressible region, and the lower section includes the thread-engaging tab(s)(or threads) and second (lower) opening. As will be appreciated, the invention encompasses all possible combinations of parts having a cap-engaging region, compressible region, and a region to engage the threads of the threaded portion of a needlefree connector, with the proviso that the final combination be capable of being compressed to provide cap-engaging and -disengaging functionality such that when corresponding structures on the cap and inner body are engaged, the cap and inner body can rotate together about their central axes, and when the corresponding structures on the cap and inner body are disengaged by compression of the compressible region, a user can rotate the cap about its central axis in relation to, or independently from (i.e., the cap spins while the resilient inner body does not), the resilient inner body.

In embodiments of the invention where the resilient inner body is made from two or more parts, those parts, once assembled, preferably are mechanically connected such that they, too, move in unison, for example, when the cleansing and capping device of which they are a part is threaded onto the threaded portion of a needlefree connector, for example, a needlefree connector. Any suitable mechanical lock, and corresponding set of mechanical structures, can be used to link such parts together.

In some preferred embodiments, the inner housing or resilient inner body also includes a sealing member (i.e., seal) configured to provide a fluid tight seal between the cleansing and capping device of the invention and an NFC connected thereto. The seal is preferably disposed in a channel formed in the inner surface of the wall of the resilient inner body proximate to the second (lower) opening, typically below the thread-engaging tab(s)(or threads).

Each device of the invention also includes a outer housing or cap in operable association with the inner housing such as a resilient inner body. A cap typically includes an outer cavity formed by a curved outer wall that is joined to a top portion, preferably about the top portion's periphery, and a preferably concentric central matrix well that extends from the top's inner surface into the outer cavity. The wall forming the matrix well is spaced from the cap's outer wall to form a resilient member housing that can be accessed through an opening created by the gap between the cap's wall and the matrix well wall. In many preferred embodiments, the inner surface of the cap's outer wall includes one or more engaging or locking structures (e.g., teeth) designed to releasably engage (i.e., corresponding structures can be engaged and disengaged, as desired) complementary structures in the cap-engaging region of the resilient inner body. In other embodiments, the cap's engaging or locking structure(s) is (are) disposed on the outer surface of the matrix well wall, which locking structure(s) is (are) designed to releasably engage complementary structures in the cap-engaging region on the inner wall of the resilient inner body. Engagement of cap's engaging or locking structure(s) with those in the cap-engaging region of the resilient inner body allows a user to rotate the cap and resilient inner body in unison, for example, as a capping and cleansing cap is screwed onto the threaded portion of a needlefree connector to be cleansed and/or capped. Once the device is releasably secured to a needlefree connector, the cap's locking structure(s) can be disengaged from those in the cap-engaging region of the resilient inner body, thereby allowing a user to rotate the cap about its central axis in relation to the resilient inner body. In some embodiments, the cap may include one or more vents to allow fluid and/or air from inside the device to escape as the cleansing and capping device is secured to a needlefree connector, while in other embodiments, no vent(s) is(are) provided.

The cleansing and capping devices of the invention also include a compressible cleansing matrix in the outer housing's matrix well. As will be appreciated, in many embodiments the compressible cleansing matrix can itself act as a resilient body or spring. The compressible cleansing matrix can be, for example, an open-cell foam. The cleansing matrix is preferably secured to an inner surface of the outer housing so as to limit or restrict its rotation independent of the outer housing during outer housing rotation. The compressible cleansing matrix is configured to contact and cleanse one or more surfaces of a needlefree connector that contacts the matrix upon a needlefree connector's association with a cleansing and capping device of the invention. The compressible cleansing matrix attached to or otherwise associated with the outer housing can be axially compressed (i.e., compressed along the central axis of the outer housing's matrix well) upon insertion of a needlefree connector into a cleansing and capping.

Because the needlefree connector surface(s) to be cleansed may be contaminated with microorganisms that form a biofilm (i.e., a matrix of microorganisms and extracellular material attached to a surface, which enables the microorganisms, typically bacteria and/or fungi, to adhere to a surface and carry out certain biochemical processes), the compressible cleansing matrix preferably has sufficient mechanical integrity when compressed to allow its use to disrupt any biofilm that may be present on a surface of the needlefree connector that is contacted by the cleansing matrix. Disruption of biofilm can occur by rotating, twisting, or otherwise moving a then-compressed cleansing matrix in relation to the needlefree connector (e.g., a needlefree medical valve), for example, by rotating the second (e.g., outer or upper) housing (to which the compressible cleansing matrix is attached or otherwise associated or retained) in relation to the first (e.g., inner or lower) housing (e.g., a resilient inner body) of the cleansing and capping device and the needlefree connector to which inner housing is releasably attached. The resulting friction between the compressed cleansing matrix and surface of the needlefree connector disrupts the biofilm, thereby cleansing, and preferably sterilizing, the needlefree connector. Leaving the cleansing and capping device secured to (i.e., capping) the needlefree connector after such cleansing will limit, and preferably preclude, biofilm regrowth and/or the microbial recolonization of cleansed surfaces (which remain in contact with the compressible cleansing matrix) of the needlefree connector.

In preferred embodiments, the compressible cleansing matrix includes one or more cleansing reagent species dispersed therein, preferably at the time the device is manufactured, although in some embodiments, the cleansing reagent may be dispersed into the matrix just prior to the matrix coming into contact with a needlefree connector. In embodiments of the latter sort, the cleansing reagent is preferably housed in the body of the cleansing and capping device in a reservoir configured to rupture upon association of a needlefree connector for cleansing. Such a reservoir can be disposed between the matrix and needlefree connector, or, more preferably, between the rotatable cap and compressible cleansing matrix. Preferred cleansing reagents include antimicrobial reagents such as isopropyl alcohol, chlorhexidine, and silver ions. In some embodiments, the cleansing and capping device of the invention will include a valve or opening to allow liquid in the cleansing reagent to evaporate.

In some preferred embodiments, the compressible cleansing matrix is comprised of two or more components. In some of such embodiments, one portion of the matrix is attached to the inner surface of the outer housing and another portion is secured to the inner surface of the wall forming the matrix well. If present, the portion of the compressible cleansing matrix secured to the inner surface of the matrix well wall is preferably configured to radially compress upon association with a needlefree connector to be capped and cleansed. When a cleansing matrix is comprised of two or more components, the matrix components may be made from the same of different material(s).

As described, the central matrix well is adapted to receive the compressible cleansing matrix. The surface(s) of the central matrix well in contact with the matrix preferably includes one or more retaining structures to retain the compressible cleansing matrix so as to link its rotation or movement to that of the cap, particularly when the engaging structures of the cap and resilient inner body are disengaged so as to allow cap rotation during a needlefree connector cleansing procedure. Such retaining structures include ridges and other protrusions from the surface of central matrix well in contact with the compressible cleansing matrix. An adhesive can also be used to adhere that portion of the compressible cleansing matrix to a desired position in the matrix well.

In various embodiments, the outer surface of the second (e.g., outer or upper) housing of a cleansing and capping device according to the invention includes one or more grip-enhancing structures or coatings, e.g., a plurality of vertical ridges, ribs, bumps, or the like (including combinations of such structures and/or coatings). Such grip-enhancing structures or coatings facilitate a user's grasp of the body of a cleansing and capping device between her/his fingers, which can be helpful not only during insertion and removal of a needlefree connector from the capping cleansing device, but also during the cleansing process, where the user rotates one housing in relation to another housing in order to scrub and thereby clean/cleanse the surface(s) of the inserted needlefree connector with the compressible cleansing matrix.

The housings of the devices of the invention can be made from any suitable material or combinations of different materials. Plastics are particularly preferred. The material(s) used to manufacture the outer housing may be the same or different as the material(s) used to produce the inner housing.

The first (e.g., inner or lower) and second (e.g., outer or upper) housings and their various components are preferably formed as a single, integral unit during manufacturing (e.g., by injection molding). The first and second housings can be manufactured by any suitable process, including extrusion, injection molding, and additive manufacturing (e.g., 3D printing). After manufacturing, the first (e.g., inner or lower) housing is operably attached to or connected with, for example, by insertion into, the second (e.g., outer or upper) housing to form a first and second housing assembly ready to assemble into a complete cleansing and capping device of the invention. For securing the first and second (e.g., inner (or lower) and outer (or upper)) housings together as a functional subassembly that can transition between engaged and disengaged configurations to provide for unitary or independent rotation of the second (e.g., outer or upper) housing in relation to the first (e.g., inner or lower) housing, any suitable retaining structure, or group of structures, that provides for movement, i.e., rotation, of the second (e.g., outer or upper) housing in relation to the first (e.g., inner or lower) housing can be used. Such structures include attachment mechanisms such as “snap-fit” mechanisms where interacting parts are sufficiently flexible and have preferably have tapered surfaces to facilitate assembly.

A compressible cleansing matrix can be positioned in the matrix well before or after a first (e.g., inner or lower) housing and second (e.g., outer or upper) housing are operably associated or assembled. In some embodiments, a suitable adhesive is used to securely adhere the compressible cleansing matrix, or, if the matrix comprises two or more parts, its various portions, to one or more inner surfaces of the second outer housing's matrix well. In some embodiments, the surface of the matrix well that contacts the compressible matrix includes one or more structures (e.g., splines, ribs, posts, etc.) to assist in securely retaining the matrix in the well, thus ensuring that it moves in conjunction with the cap when the outer housing is rotated during a cleansing procedure.

In preferred embodiments, the cleansing and capping devices of the invention include a removable lid or seal attached to one or more housing to seal the device, thus separating the interior spaces and structures of the device from the external environment. Such a lid or seal prevents exposure of the device's interior, including, for example, its inner (e.g., lower) housing and compressible cleansing matrix, to the environment until the seal is removed, typically by a healthcare worker just prior to her/his use of the cleansing and capping device to cap and then, if desired, to clean/cleanse the needlefree connector (e.g., needlefree medical valve) to which it is connected. In preferred embodiments, such cleansing substantially disrupts any biofilm that may exist on surfaces contacted by the compressible cleansing matrix. If desired, the cleansing and capping device can be left in place (typically after cleansing the needlefree connector attached thereto) in order to cap the needlefree connector until it is further accessed, thereby minimizing exposure of capped exterior surfaces of the connector to potential pathogen contamination (and biofilm formation) from the surrounding environment. Seals are typically installed during manufacture of a cleansing and capping device of the invention. In those embodiments where the cleansing and capping devices are sterilized during manufacture (e.g., by irradiation, exposure to ethylene oxide, etc.), seals are preferably applied prior to sterilization.

Other aspects of the invention concern methods of cleansing and/or capping needlefree connectors using a cleansing and capping device according to the invention. In many embodiments, such methods typically involve transitioning the inner and outer housings from an engaged to a disengaged configuration after the device has been connected to a needlefree connector as to allow the outer housing to spin or rotate in relation to the inner housing. In embodiments where the second (outer) housing is downwardly compressed to engage the first (inner) housing, such transitioning facilitates contact between the device's compressible cleansing matrix and the associated surface(s) of the needlefree connector. Spinning or rotation of the second (e.g., outer or upper) housing in relation to the first (e.g., inner or lower) housing, and the associated surface(s) of the needlefree connector, allow those surfaces to be scrubbed, thereby cleansing them. Preferably, such cleansing methods provide for the disruption of any biofilm present on the surface(s) of the needlefree connector associated the cleansing and capping. And in those embodiments where the compressible cleansing matrix contains one or more antimicrobial reagents, microbes and pathogens present in such biofilm and/on on such surface(s) are destroyed or rendered nonviable.

Herein, the compressible cleansing matrix of a cleansing and capping device of the invention comprises one or more cleansing reagent species dispersed in a substrate. The cleansing matrix substrate can be any substance that can conform, mold, or compress in a manner that enables the effective friction-based cleansing of the site or portion of the needlefree connector to be cleansed, including the top surface of the site, side surface, and any threads or grooves, if present, and provide the cleansing reagent at least at a surface level. Examples of the compressible cleansing matrix include cotton, open or closed cell foam such as polyethylene foam, or other substance that can hold or carry the cleansing reagent.

In some embodiments, the cleansing reagent species is(are) dispersed in or otherwise combined with the compressible cleansing matrix during the process used to manufacture the cleansing and capping, while in other embodiments, the device is configured such that the cleansing reagent(s) is(are) released for dispersion into the compressible cleansing matrix post-manufacture, but when or prior to the time the matrix is brought into contact with the needlefree connector to be cleansed. The cleansing reagent can be any chemical, substance, or material that cleans the site of bacterial or even viral microorganisms, biofilm, etc., or any carrier that contains such chemical, substance or material. Examples of cleansing reagents include isopropyl alcohol, chlorhexidine, chlorhexidine digluconate, povidone-iodine, hydrogen peroxide, soap, and hydrochloric acid, silver ions and salts (e.g., silver acetate, silver lactate, silver nitrate, etc.), etc.

In accordance with the invention, a cleansing reagent comprises an active ingredient capable of cleansing a surface of a needlefree connector. Any active ingredient that can be used effectively to rapidly cleanse a medical fitting or medical line connector (e.g., a needlefree connector) can be adapted for use in practicing the invention, and are generally classified as antibacterial and/or antifungal reagents, antiseptic or antimicrobial reagents, wide spectrum disinfectants, and/or parasiticides, as well as combinations of such reagents. Particularly preferred are biocompatible cleansing reagents, as the devices of the invention are intended for human and/or veterinary use, including alcohols, antibiotics, oxidizing reagents, and metal salts. Representative examples of such active ingredients include bleach, chlorhexidine, ethanol, isopropyl alcohol, hydrogen peroxide, sodium hydroxide, and an iodophor dissolved or otherwise dispersed in a suitable solution, suspension, or emulsion. Other active ingredients having suitable cleansing effects can also be used. These include alcohols (e.g., ethanol, benzyl alcohol, isopropyl alcohol, phenoxyethanol, phenethyl alcohol, etc.); antibiotics (e.g., aminoglycosides, such as amikacin, apramycin, gentamicin, kanamycin, neomycin, netilmicin, paromomycin, rhodostreptomycin, streptomycin, and tobramycin; bacitracin; chloramphenicol; erythromycin; minocycline/rifampin; tetracycline; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins; etc.), quaternary ammonium chlorides; quaternary ammonium carbonates; benzalkonium chloride; chlorinated phenols; fatty acid monoesters of glycerin and propylene glycol; iodine; iodine containing compounds, such as 3-iodo-2-propynyl butyl carbamate (IPBC); iodophors, such as povidone-iodine (Betadine 100%, which contains providine iodine as the active ingredient); hydantoins, such as dimethylhydantoin and halogenated hydantoins; isothiazolinones; parabens, such as methylparaben, ethylparaben, and propylparaben; chloroxylenol; chlorhexidine and its salts; chlorhexidine/silver-sulfadiazine; chlorhexidine acetate; chlorhexidine gluconate (e.g., Hibiclens); chlorhexidine hydrochloride; chlorhexidine sulfate; benzoic acid and salts thereof; benzalkonium chloride; benzethonium chloride; methylbenzethonium chloride; chlorobutanol; sorbic acid and salts thereof; imidazole antifungals (e.g., miconazole); butocouazole nitrate; mafenide acetate; nitrofurazone; nitromersol; triclocarban; phenylmercuric nitrate or acetate (0.002%); chlorocresol; chlorbutol; clindamycin; CAE (Anjinomoto Co., Inc., containing DL-pyrrolidone carboxylic acid salt of L-cocoyl arginine ethyl ester); cetylpyridinium chloride (CPC) at 0.2%, 0.02%, and 0.002% concentrations; 9.8% isopropyl alcohol; 1% ZnEDTA; mupirocin; and polymyxin (polymyxin b sulfate-bacitracin). Additionally, other useful compounds and compositions include Miconazole, Econazole, Ketoconazole, Oxiconizole, Haloprogin, Clotrimazole, butenafine HCl, Naftifine, Rifampicin, Terbinafine, Ciclopirox, Tolnaftate, Lindane, Lamisil, Fluconazole, Amphotericin B, Ciprofloxecin, Octenidine, Triclosan (2,4,4′-trichloro-2′-hydroxydiphenyl ether), Microban (5-chloro-2phenol (2,4 dichlorophenoxy). Useful metals include silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine. Cleansing reagents are often compositions that comprise the desired active ingredient(s) in admixture with other ingredients, such as carriers and liquid solvents.

The particular active ingredient(s) selected as a cleansing reagent for a given application will be compatible with the compressible cleansing matrix and material(s) used to form the housings and other components of the particular device. In some embodiments, the cleansing reagent is dispersed in the compressible cleansing matrix after the matrix is formed. For example, a cleansing reagent can be dispersed by saturating or supersaturating a compressible cleansing matrix during manufacture of the device, preferably before it is sealed. In other embodiments, the cleansing reagent can be dispersed during the process used to manufacture the compressible cleansing matrix. As will be appreciated, the materials used to prepare the cleansing reagent should be compatible with the constituent or constituents that comprise the compressible cleansing matrix such that the substrate does not appreciably degrade or otherwise suffer loss of structural integrity prior to being used to cleanse a needlefree connector (e.g., a needlefree medical valve). Similarly, the cleansing reagent should be biocompatible, such that it will not harm a patient in the event of contact or should some amount of the cleansing reagent be admitted into the fluid-carrying portion of a needlefree medical valve, as well as with materials used to form needlefree medical valves (or other needlefree connector).

In preferred embodiments, the material used to form the compressible cleansing matrix is any suitable absorbent, compliant, pliable, resilient, fibrous, or porous material, or combination of materials, than can be wetted and/or impregnated with a cleansing reagent and which can easily and readily adapt to complex surface contours (e.g., luer threads, concave and convex surfaces, flanges, etc.) likely to be engaged upon contact with, for example, a needlefree medical valve to be cleansed. Such materials include those that are synthetic or naturally occurring, and they may be of homogeneous or heterogeneous composition. Preferred synthetic materials include fibrous, foam (e.g., a felted foam), and gel compositions, particularly those having directionally oriented natural or synthetic fibers, or combinations thereof. Preferred naturally occurring materials useful as substrates include fibrous naturally occurring materials, including plant-derived materials such as cotton and paper products, as well as animal-based fiber products such as wool. Other preferred natural materials are sponges.

As will be appreciated, in order to achieve the desired cleansing effect, a compressible cleansing matrix, or the component part(s) thereof designed to contact a needlefree connector such as a needlefree medical valve, preferably are made of a material (or combination of materials) that allow the cleansing element to thoroughly cleanse surfaces of needlefree connectors such as needlefree valves or luer access devices, particularly those surfaces that are exposed to air and thus are at risk for contamination with infectious or pathogenic reagents, and biofilms containing the same, and are also intended to form part of the fluid flow path for fluids to be introduced into a patient, for example, IV solutions, medications, blood and blood products, etc.

Preferably, the material used to produce the compressible cleansing matrix should be sufficiently compliant to allow the compressible cleansing matrix to deform under the pressures experienced during normal use in order to allow it to conform to the external structures present on the surface(s) of the needlefree connector to be cleansed. This assures intimate, cleansing contact between the compressible cleansing matrix and at least those exposed surfaces of, for instance, a needlefree connector designed to come into contact with fluid entering the valve, such as IV fluids. In addition, the compressible cleansing matrix preferably allows for the retention of a liquid cleansing reagent, for example, in capillary spaces, in the void volume of foams, sponges, etc. The compressible cleansing matrix may also be engineered such that it includes cleansing reagents such as silver ions and/or other suitable materials.

Preferred natural materials from which compressible cleansing matrices can be formed include those derived from cotton and naturally occurring sponges. As those in the art appreciate, processed cotton fibers are composed almost entirely of the natural polymer cellulose. In such fibers, 20-30 layers of cellulose are coiled into a series of spring configurations, which makes the fibers absorbent and gives them a high degree of durability and strength. For example, woven cotton sheets, as are often used in the manufacture of sterile cleansing pads that are then saturated with a 70% isopropyl alcohol (IPA) solution, can be used as substrates for cleansing elements according to the invention. Any suitable configuration may be used. For example, a woven cotton sheet can be cut into numerous similarly sized pieces, each of which can be used as a substrate. In many embodiments, after attachment to the inside surface of a layer of the container (e.g., through the use of an adhesive, double-sided, tape, etc.), the matrix is ready for the addition of a suitable cleansing reagent. Alternatively, cotton fibers can be spun onto the inside surface of the cap. Other fibers, be they naturally occurring, synthetic, or combinations of natural and synthetic materials, having similar properties can also readily be adapted for use as compressible cleansing matrices.

Another class of materials for compressible cleansing matrix fabrication is directionally oriented fibrous materials. These include, without limitation, materials comprised of cellulose fibers, glass fibers, and polyester fibers, as well as materials comprised of combinations of two of more of these and/or other materials. Such bonded synthetic fibers use capillary action to precisely absorb, retain, transfer, and/or release liquids or vapor in desired amounts. A broad range of synthetic polymers can be used to form the fibers, and, if desired, they may be treated for functional purposes, for example, to contain a cleansing reagent dispersed therein, to provide a vapor barrier or other coating over a portion of the product's surface, etc. The geometric shape of these materials can also be customized for particular applications, thereby permitting easy integration into substrate configurations having the desired device thickness, widths, length, diameter, etc.

Other representative classes of materials suitable for use as compressible cleansing matrices include gel-forming polymers and foams such as agarose, agar, polyacrylamide, and other synthetic porous materials that can be formed into layers, sheets, columns, or other shapes compatible with practicing the invention. Representative gelatinous materials include hydrogels (i.e., cross-linked polymers that absorb and hold water), particularly those made from agarose, (2-hydroxyethyl)methacrylate and its derivatives, and synthetic carbohydrate acrylamides.

Still other classes of materials include porous polymer sponges. Such sponges can be formed from any suitable material, including polyethylene, polypropylene, olytetrafluoroethylene, polyvinylidine difluoride, polynitrile, and polystyrene. Many such porous polymer sponges are commercially available in a wide variety of shapes, pore density and size, etc. Additionally, polymer sponges can be made by polymerizing appropriate monomers according to conventional foam forming techniques. In general, sponges have an open pore structure to allow movement of a solvent such as a liquid cleansing reagent. The sponge surface should include open pores to provide entry of liquid cleansing reagents (e.g., alcohol, iodine-containing solutions, etc.), and, as with other materials used to form matrices, the particular material chosen is preferably inert, i.e., not reactive with components of the cleansing reagent, the body of the cleansing and capping, or the materials used to produce needlefree connectors such as needlefree medical valves.

Surgical foams are another preferred class of materials that can be used to make compressible cleansing matrices. The materials can be natural or synthetic, as desired. Suitable foams include rubber latex, polyurethane, polyethylene and vinyl foams. Preferably, such foams are made from any suitable biocompatible polymer, for example, polyvinyl alcohol (PVA) or polyurethane. One preferred foam material is Microbisan™, a hydrophilic polyurethane foam that is impregnated with silver ions (Lendell Manufacturing, St. Charles, MI). Preferably, such foams are highly absorbent and thus suitable for use with liquid cleansing reagents. In other embodiments, the material used to form the foam is well-suited for dispersion of a dry cleansing reagent, such as silver ions. Again, it is preferred that foam materials, if used to as a substrate, be inert. Also, they are preferably sufficiently flexible to conform to the variety of different shapes and surface configurations (e.g., double seal fluid access points, luer threads, etc.) encountered in the field given the multitude of medical valve shapes, sizes, and configurations. In this way sufficient contact between the cleansing surface(s) of the cleansing and capping device and the surface(s) of the needlefree connector to be cleansed can be ensured. Another advantage of some synthetic foams (as well as certain other polymeric materials from which substrates may be formed) is that they can easily be injected in a desired volume into a shell or housing during manufacture, after which they expand to assume the desired substrate size, density, porosity, etc.

Furthermore, compressible cleansing matrices can include chemicals to indicate a functional change therein, for example, by using a color change to signal a change from a wet to a dry state, or, alternatively, that the matrix material has been properly wetted with a liquid cleansing reagent dispersed into the substrate by a health care worker just prior to use, as opposed to during manufacture of the device. Thus, depending on the system used, a color change in the matrix could be used to indicate that the cleansing reagent in the compressible cleansing matrix has evaporated prior to use and thus the particular cleansing device should not be used, perhaps due to a leak in the cleansing and capping device's storage container. Alternatively, when, for example, a colored liquid cleansing reagent is used, the user can visually confirm dispersion of the reagent in the matrix by assessing whether the colored cleansing reagent is dispersed throughout the matrix. When colored cleansing reagents are used, it is preferred that the material(s) use to make the resilient inner body and/or cap of the cleansing and capping device be clear or translucent, or include one or more clear or translucent windows, in order to allow easy visualization of any color change prior to or during use of the cleansing and capping.

Capping and cleansing devices of the invention and their constituent parts (e.g., first and second housings, compressible cleansing matrix, disinfectant, sealing ring (if present), lid/seal, etc.) can be made from any suitable material(s) and assembled using any suitable process.

Preferably, at least a portion of the outer surface(s) of a cleansing and capping device of the invention (e.g., the outer housing, the upper and lower housings, etc.) that is (are) intended for grasping by a user has a non-slip surface, i.e., one having a high coefficient of friction so that when the outer portion of a cleansing and capping device is held in a user's fingers and positioned to cleanse a needlefree connector so that the housing(s) can be rotated in relation to the inner housing and needlefree connector with minimal or no slippage between the device and the user's fingers (gloved or ungloved). Examples of such non-slip (or high friction) surfaces include those having ridges, valleys, dimples, bumps, or other features designed to enhance friction, as well as combinations of two or more of such features. Such features can be introduced into a device's outer surface(s) as part of the manufacturing process. Alternatively, a non-slip coating can be applied to one or more of the outer surfaces of the second (e.g., outer) housing, for example, while in embodiments where both the first and second housings are designed to be grasped by a user, for example, to attach the device to a needlefree connector or to cleanse the connector's valve surface, some or all of the exterior surfaces of such housings will advantageously include such features.

In general, the cleansing and capping devices of the invention are provided to users in a sealed, sterile manner. If desired, labeling information, logos, artwork, manufacturing, and/or regulatory data (e.g., lot number, expiration or “use by” dates, etc.) may also be printed or otherwise applied to individual cleansing and capping devices. In addition, information such as a bar or QR code (to allow use of the device to tracked, for example) may also be included on individual cleansing and capping devices.

As will be appreciated, cleansing devices may be packaged individually or in groups of two or more units as kits, which can further include instructions for use of the cleansing and capping device(s), as well as other information, logos, artwork, manufacturing, and/or regulatory data.

In preferred embodiments, packaged cleansing and capping devices are sterilized using a suitable process, such as irradiation. In a particularly preferred practice, the cleansing and capping devices of the invention are sterilized as part of the manufacturing process. Here, “sterilization” refers to any process that effectively kills or eliminates transmissible reagents, e.g., bacteria, viruses, fungi, prions, spores, etc. that may be present in any component of a device according to the invention. In preferred embodiments, sterilization can be achieved by heating, chemical treatment, irradiation, and other processes. Indeed, any sterilization process compatible with the materials used to make the cleansing and capping device can be employed. A particularly preferred sterilization process is an irradiation process. Such processes include irradiation with x-rays, gamma rays, or subatomic particles (e.g., an electron beam). In general, when a sterilization process is used in the context of the invention, the process is employed on a cleansing article after it has been sealed and/or packaged. Chemical sterilization processes can also be used, for example, sterilization using ethylene oxide (EtO).

The invention also concerns methods of using the instant single-use cleansing and capping devices of the invention. Such methods include using the devices to cleanse and, if desired, cap needlefree connectors such as needlefree connectors, luer access devices, and the like. To perform such methods, the portion of a needlefree connector to be cleansed is threaded into the central bore of a first (e.g., inner or lower) housing of a cleansing and capping device, typically after the user (e.g., a nurse) removes a seal (or “lid”), advantageously a peelable seal or lid, that spans the opening in the device. Such insertion brings the site of the needlefree connector into contact with (i.e., brought into cleansing association with) the compressible cleansing matrix portion(s) of the device. In preferred practice, once the compressible cleansing matrix is in contact with the surface(s) of the needlefree connector to be cleansed, the second (e.g., outer) housing automatically disengages the engaging elements in the first (e.g., inner) and second (e.g., outer) housings to allow rotation of the second housing in relation to the first housing and needlefree connector previously releasably connected to cleansing and capping device. Such contact and cleansing action can be for any desired period, with periods of about one second to about ten to twenty seconds being particularly preferred.

After cleansing, the needlefree connector can be removed from the cleansing and capping device, after which the cleansing and capping device may be discarded. Alternatively, after cleansing, the cleansing and capping device can be left attached to the needlefree connector, capping a portion thereof until such time as access to the needlefree connector is desired, “passively” capping it and protecting it from contamination. At that time, the cleansing and capping device can be removed and discarded. If desired, just prior to removal, a cleansing process can be repeated.

After removal of a cleansing and capping device from a cleansed needlefree connector, a fluid-containing medical reservoir (e.g., a syringe containing a medication, an IV bag, etc.) may be immediately connected to the cleansed needlefree connector. In preferred embodiments where the cleansing reagent is a solution, the surface(s) of the needlefree connector is preferably allowed to dry (or is(are) dried, for example, by wiping with a sterile, absorbent cloth or wipe, which cloth or wipe may be dry or wetted with a volatile, compatible solution such as 70-100% alcohol) prior to connecting the needlefree connector to a fluid reservoir. In preferred practice, such cleansing methods result in at least a 2-fold, 5-fold, or 10-fold or more reduction in microorganism contamination on the accessible surface(s) that have been cleansed. Even more preferably, the level of reduction may exceed a 100-fold, a 10³-fold, a 10⁴-fold, a 10⁵-fold, a 10⁶-fold, 10⁷-fold, 10⁸-fold, 10⁹-fold, or greater reduction in microorganism contamination on the accessible fitting surface.

In addition to methods for cleansing accessible surfaces of luer access devices and the like, the devices of the invention provide methods of reducing infection risk in a patient connected to devices, such as a peripheral IV line, a central IV line, or a peripherally inserted central catheter, configured for delivering fluids directly into the patient's blood stream. The risk reduction afforded by the devices of the invention may vary depending upon many factors, such as patient age and condition, the condition being treated, the location where medical services are being delivered, patient density, the level of contaminating microorganisms in the environment, the quality of air handling equipment in the medical facility, the degree of training of medical personnel charged with cleansing the access device, the method(s) used to periodically cleanse the medical fitting, intervals between cleansing procedures, the particular configuration of the cleansing and capping, the particular configuration of the needlefree connector, whether the cleansing and capping device is left on the cleansed site of the needlefree connector in order to provide capping, etc. Risk reduction can be established using any suitable method, for example, by assessing HAI frequency in the presence and absence of using cleansing devices according to the invention. Reductions of HAI infection risk of 1-100% or more, including up to 1000% or more, are envisioned through use of cleansing and capping s according to the invention. As will be appreciated, reductions in infection risk (e.g., HAI risk) will translate to improved patient outcomes (through reduced morbidity and mortality) and reduced expenditure on treating HAI's.

### Representative Embodiments

To further illustrate and describe certain preferred, representative embodiments of the invention, the reader is directed to the appended drawings, FIGS. 1-3, which illustrate various particularly preferred embodiments of the cleansing and capping s of invention. A description of these preferred, representative embodiments follows.

FIG. 1 shows three drawings, A, B, and C, of a representative cleansing and capping device of the invention, its constituent parts (A), and two views of the device associated with a needlefree connector (B and C).

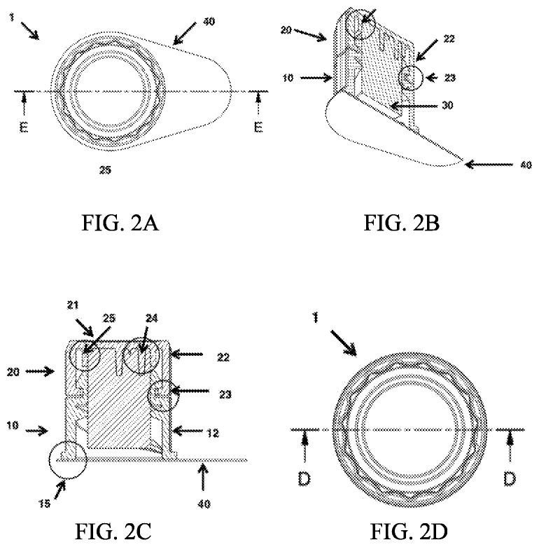

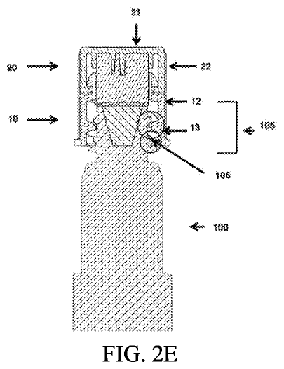

FIG. 2 contains five drawings, A-E. FIGS. 2A-C show different views of an assembled, sealed representative cleansing and capping device of the invention, and FIGS. 2D and 2E show that device operably connected to a needlefree connector.

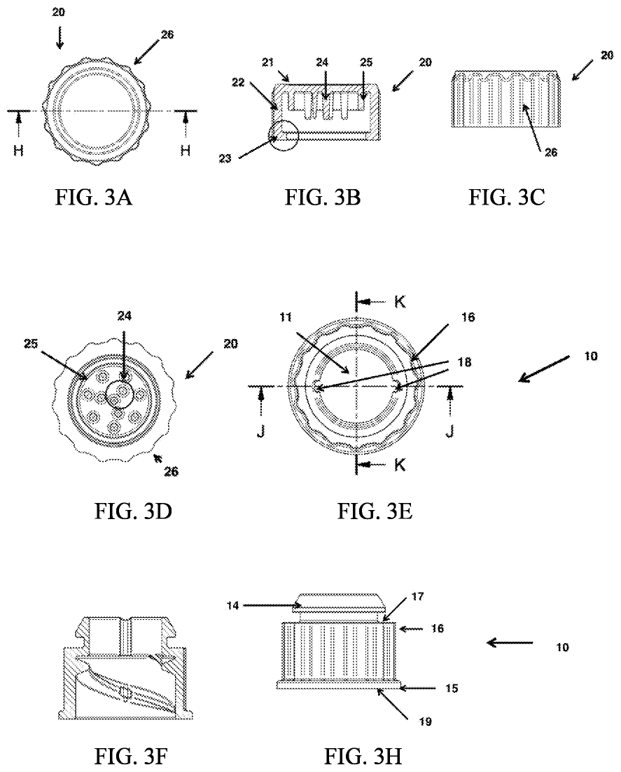

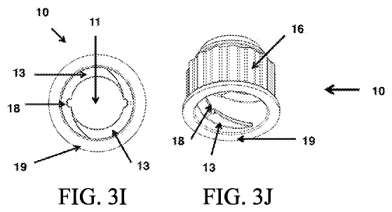

FIG. 3 contains 10 drawings, A-J, that show four views of the second (upper) housing (A-D) and six views (E-J) of the lower housing of a representative cleansing and capping device of the invention.

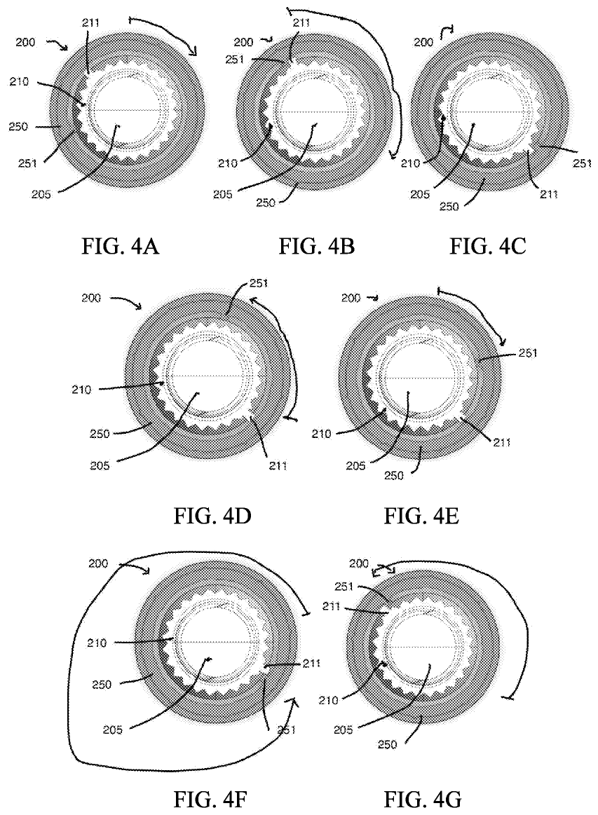

FIG. 4 contains 7 drawings, A-G, that show seven cross-section views looking up from below at the inner and outer (second and first, respectively) housings of a representative cleansing and capping device of the invention threaded onto an needlefree connector.

FIG. 1 has three drawings that show different views of representative cleansing and capping device of the invention (1) that includes a second (upper) housing (20), a first (lower) housing (10), a compressible foam cleansing matrix (30) that is secured in or to the underside of the top of the second (upper) housing and protrudes though an upper opening in top of the first (lower) housing (10), and a seal (or “lid”) attached to the bottom surface of the lower housing (10). FIG. 1A is an exploded view showing these components. FIG. 1B shows the device (1) attached to a representative needlefree connector (100). In FIG. 1B, the device does not include the seal (40) as it is to be removed from the device (1) prior to attachment to a needlefree connector (100), and the foam (30) is not visible when the device (1) is functionally attached to an NFC (100). In this embodiment, the upper housing (20), to which the foam (30) is secured, can freely rotate about its central axis, thereby also causing the foam (30) to rotate. Such rotation allows the device (1) to be used to “actively” scrub (disinfect or cleanse) the valve portion (not visible) of the needlefree connector. FIG. 1C shows a substantially isometric view of the assembly shown in FIG. 1B.

FIG. 2 shows one view from above (FIG. 2A) of an assembled version of the device (1) depicted in FIG. 1A and four cutaway drawings (FIGS. 2B-2E; defined by planes E-E or DD) of the assembly shown in FIGS. 1B and 1C. As shown, the lower housing (10) includes a sidewall (10) that defines a cavity having on its lower inner surface threads (13) complementary to those on the threaded valve portion (105) of the NFC (100). The cavity has a bottom opening sized to accommodate the threaded valve portion (105) of the NFC (100). The bottom surface of the lower housing (10) defines a sealing flange (15) to which the seal (40) can be secured, for example, through the use of heat and/or pressure sensitive adhesive layered on the flange-facing surface of the seal (40). The top of the first (lower) housing (10) also includes an upper opening, opposite the bottom opening, through which the compressible cleansing matrix (30) extends into the cavity in the first housing. When the device (1) is threaded onto and securely attached to an NFC (100), the compressible cleansing matrix (30) becomes compressed (see, e.g., FIG. 2E).

As shown in FIGS. 2B and 2C, in this embodiment of the invention the second (upper) housing (20) includes a circumferential matrix well (25) that extends from the inner surface of the top (21) of the second housing (20). The matrix well (25) is sized to accommodate and retain the compressible cleansing matrix (30), here, a foam cylinder. A set of matrix retaining elements (24) located within the matrix well (25) and also extending from the inner surface of the top (21) of the second housing (20) provides for further support and retention of the foam (30).

In the device embodiment illustrated in FIGS. 1-3, the second (upper) housing (20) is joined to the first (lower) housing via a complementary connection flange (23) located at the bottom of the sidewall (22) of the second housing (20) and a connection flange channel (17) disposed on the retaining element (14) of the first (lower) housing (10), although any suitable set of retaining structures or elements disposed on facing surfaces of the first and second housings that allow for the housings to rotate about their central axes (here, a common central axis), preferably independently, can be used and are within the scope of the invention. In related embodiments (not shown), one or more fluid tight seals between the first and second housings (10, 20) can be included. Advantageously, such seals, when included, do not markedly impact the ability of the housings (10, 20) to rotate in relation to each other. Other elements may also be included, for example, structures that provide haptic feedback to users of the device (1) when it is attached to an NFC (100).

The ten drawings of FIG. 3, FIGS. 3A-3J, show four views of the second (upper) housing (A-D) and six views (E-J) of a lower housing of a representative cleansing and capping device of the invention. In this Figure, grip-enhancing elements (16, 26) are shown on both first and second housings (10, 20). Also, in FIGS. 3E, 3F, 3G, 3H, and 3I, fluid paths (18) are illustrated on the inner surface of the sidewall (12) of the first housing (10) that extend from the top of the first housing (10) into the housing's threaded lower cavity. In the embodiment illustrated here, two opposed fluid paths (18) are shown, although one or three or more spaced fluid paths can be included in other embodiments, if desired. Such fluid paths provide a channel though which liquid disinfectant can flow onto the threaded region of the valve portion (105) of an NFC (100), particularly as (or after) the compressible cleansing matrix (30) is compressed by thread-driven insertion of an NFC (100) into the cavity of the first housing (10) of the device (1).

FIG. 4 contains 7 drawings, A-G, that show seven cross-section views looking up from below at the inner and outer housings (210, 250) (second and first housings, respectively) of a representative cleansing and capping device (200) of the invention threaded onto a needlefree connector (205).

As with a number of other embodiments of the invention, the embodiment represented in FIG. 4 contains a flexible outer housing (250) that can be “squeezed” by a user such that the outer housing (250) can then engage the inner housing (210) so as to allow the inner and outer housings to rotate in unison, for example, during attachment and removal of the device (200) from a needlefree connector (205). In the embodiment depicted in FIG. 4, the inner and outer housings include complementary tabs (211, 251, respectively) that, even in the absence of “squeezing” the outer housing (250) to engage the inner housing (210), when the outer housing (250) is rotated such that its inner housing engaging tab (251) contacts or engages the corresponding outer housing engaging tab (211) that protrudes from the exterior surface of the inner housing (210), the inner and outer housings (210, 250) then move together if a user continues to rotate the outer housing (250) in the same direction. In some embodiments, a plurality of inner and outer engaging tabs (e.g., 2, 3, 4, or more) (211, 251)can be included. In such embodiments, such tabs can be evenly or unevenly spaced, depending on the design parameters of the particular device (200). Such inner and outer engaging tab features or the like can assist in allowing a user to securely attach (or detach) the cleansing and capping device (200) from the needlefree connector (205).

FIGS. 4A-4C illustrate a device (200) that has been at least partially threaded onto a needlefree connector (205). See FIG. 4A. At that point, the inner and outer engaging tabs (211, 251) have yet to come into contact. Upon rotation (see arrow, FIG. 4A) of the outer housing (250) in relation to the inner housing (210) of FIG. 4B, the inner and outer engaging tabs come into contact. Further rotation of the device (200) in the same direction (see arrow, FIG. 4B), with the engaged inner and outer housings (210, 250) rotating together allow for further tightening of the device (200) onto the needlefree connector (205). See FIG. 4C.

FIGS. 4D and 4E then depict how the outer housing (250) (free from rotational engagement with the inner housing (210)) of the now-securely attached device (200) can be rotated (illustrated by the arrows) first in the direction opposite (see in FIG. 4D) that used to tighten the device (200) onto the needlefree connector (205) and then again in the direction used for device attachment (see FIG. 4E) in order to “actively” scrub (i.e., cleanse or disinfect) the valve surface (not shown) of the needlefree connector (205).

FIGS. 4F and 4G then depict how the device of the invention (200) can be removed from the connector (205). This process begins by rotating the outer housing (250) (free from rotational engagement with the inner housing (210)) in the direction opposite that used to tighten the device (200) onto the needlefree connector (205) until the inner and outer engaging tabs (211, 251) engage. See FIG. 4F. Thereafter, when rotation continues (see FIG. 4G) in the same direction, the device (200) can be unthreaded (i.e., detached) from the connector (205).

As those in the art will also appreciate, the invention envisions cleansing and capping devices that include alternative or additional structures that allow the first and second housings of a device according to the invention to engage or disengage, for example, to allow attachment or detachment from a needlefree connector (e.g., for engagement or disengagement) or to provide for “active” scrubbing (e.g., by independently rotating the outer housing in relation to the inner housing). FIG. 4 illustrates a representative example of such a device. FIG. 4 and this feature can assist in allowing a user to securely attach (or detach) the cleansing and capping device (200) from the needlefree connector (205).

Unless the context clearly requires otherwise, throughout the description above and the appended claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list, and any combination of the items in the list.

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law.

What is claimed is:

1. A cleansing and capping device for a threaded valve portion of a needlefree connector, comprising:
(a) a first housing configured for secure, removable attachment to a threaded valve portion of a needlefree connector;
(b) a second housing disposed on top of and rotatably connected to the first housing;
(c) a compressible cleansing matrix attached to the second housing and protruding into the first housing, wherein the compressible cleansing matrix rotates with the second housing; and
(d) a liquid disinfectant disposed in the compressible cleansing matrix.

2. The cleansing and capping device according to claim 1 that further comprises a removable lid to seal device from an external environment.

3. The cleansing and capping device according to claim 2 that has been sterilized.

4. The cleansing and capping device according to claim 1 wherein the first housing includes at least one fluid path disposed on an inner surface of a sidewall.

5. The cleansing and capping device according to claim 1 wherein the compressible cleansing matrix comprises a liquid disinfectant, optionally isopropyl alcohol.

6. The cleansing and capping device according to claim 5 wherein the liquid disinfectant comprises a solution of about 70% isopropyl alcohol and water.

7. The cleansing and capping device according to claim 6 wherein the liquid disinfectant further comprises chlorhexidine, optionally chlorhexidine gluconate.

8. The cleansing and capping device according to claim 1 that further comprises a seal between the first housing and the second housing.

9. The cleansing and capping device according to claim 1 wherein the compressible cleansing matrix is configured to contact at least a valve surface of the threaded valve portion of the needlefree connector when the device is threaded onto the threaded valve portion of the needlefree connector.

10. The cleansing and capping device according to claim 9 wherein the compressible cleansing matrix is configured to also contact an additional surface of the threaded valve portion of the needlefree connector when the cleansing and capping device is threaded onto the threaded valve portion of the needlefree connector.

11. The cleansing and capping device according to claim 1 wherein the first housing comprises a second housing engaging tab and the second housing comprises a first housing engaging tab.

12. An assembly that comprises a single piece of lidding on to which a plurality of cleansing and capping devices according to claim 1 are sealed prior to or after sterilization.

13. An assembly that comprises a cleansing and capping device according to claim 1 threaded on to a threaded valve portion of a needlefree connector.

14. An assembly that comprises a plurality of cleansing and capping devices according to claim 1 sealed to a single piece of lidding stock.

15. An assembly that comprises a plurality of individually sealed cleansing and capping devices according to claim 2 removably connected to a strip configured to hang from an IV pole.

16. A method of cleansing a needlefree connector, comprising:
(a) securing a cleansing and capping device according to claim 1 to a threaded valve portion of the needlefree connector; and
(b) rotating the second housing in relation to the first housing, thereby cleansing one or more surfaces of the threaded valve portion of the needlefree connector.

17. The method according to claim 16 that further comprises removing the cleansing and capping device from the threaded valve portion of the needlefree connector immediately after cleansing the one or more surfaces of the threaded valve portion of the needlefree connector.

18. The method according to claim 16 that further comprises leaving the cleansing and capping device connected to the threaded valve portion of the needlefree connector.

19. The method according to claim 18 that further comprises leaving the cleansing and capping device connected to the threaded valve portion of the needlefree connector for up to 7 days.
